(19) United States
(12) Patent Application Publication
(10) Pub. No.: US 2009/0099408 A1
(43) Pub. Date: Apr. 16, 2009

(54) METHODS AND APPARATUS FOR TREATING TINNITUS

(75) Inventors: Amir ABOLFATHI, Woodside, CA (US); John SPIRIDIGLIOZZI, San Mateo, CA (US); Reza KASSAYAN, Atherton, CA (US)

Correspondence Address: LEVINE BAGADE HAN LLP, 2483 EAST BAYSHORE ROAD, SUITE 100, PALO ALTO, CA, 94303, US

(73) Assignee: Sonitus Medical, Inc., San Mateo, CA (US)

(21) Appl. No.: 12/333,266

(22) Filed: Dec. 11, 2008

Related U.S. Application Data
(63) Continuation of application No. 11/845,712, filed on Aug. 27, 2007.
(60) Provisional application No. 60/825,099, filed on Sep. 8, 2006.

(51) Int. Cl. A61B 8/08; A61H 1/00

(52) U.S. Cl. 600/25; 601/46

(57) ABSTRACT

Methods and apparatus for treating tinnitus are described where an oral appliance having an electronic and/or transducer assembly for generating sounds via a vibrating transducer element is coupled to a tooth or teeth. Generally, the transducer may generate one or more frequencies of sound via the actuatable transducer positioned against at least one tooth such that the sound is transmitted via vibratory conductance to an inner ear of the patient, whereby the sound completely or at least partially masks or provides sound therapy for habituation of the tinnitus perceived by the patient. The one or more generated frequencies may be correlated to measured tinnitus frequencies or they may be preset.

### CROSS-REFERENCE TO RELATED APPLICATIONS

This application is a continuation of Ser. No. 11/845,712 filed Aug. 27, 2007 which claims the benefit of priority to U.S. Prov. Pat. App. 60/825,099 filed Sept. 8, 2006, each of which is incorporated herein by reference in its entirety.

### FIELD OF THE INVENTION

The present invention relates to methods and apparatus for treating tinnitus via oral-based hearing aid appliances. More particularly, the present invention relates to methods and apparatus for treating tinnitus via oral appliances which are positionable within a mouth of a patient for transmitting sound conduction through teeth or bone structures in and/or around the mouth to mask or habituate a patient to sounds or ringing typically associated with tinnitus.

### BACKGROUND OF THE INVENTION

Tinnitus is a condition in which those affected perceive sound in one or both ears or in the head when no external sound is present. Often referred to as “ringing” in the ears, tinnitus can occur intermittently or consistently with a perceived volume ranging from low to painfully high. However, the perceived volume of tinnitus can vary from patient to patient where an objective measure of tinnitus volume in one patient may be perceived as painful but in another patient the same volume may be perceived as subtle.

Generally, tinnitus can be caused by a number of sources. For instance, exposure to loud noises can lead to damage of the cilia within the inner ear. An accumulation of wax within the ear canal can also amplify a person's tinnitus condition. Other factors such as ingestion of certain medications, ear or sinus infections, tumors growing on auditory nerves, as well as trauma to the head or neck can also induce tinnitus. Additionally, a small percentage of tinnitus patients may experience a form of tinnitus known as pulsatile tinnitus where a rhythmic pulsing sound is present which is attuned to the patient's heartbeat. Such a condition may be indicative of a cardiovascular condition such as pulmonary stenosis, hypertension, hardening of the arteries, arterio venous malformations, etc.

Treatments for tinnitus vary greatly. For instance, masking therapy typically involves using a hearing aid device to introduce sounds at a level and frequency that completely or partially cover the sounds of tinnitus in a patient to provide immediate short-term relief. Another similar therapy, tinnitus retraining therapy (TRT) or habituation, is a form of combination treatment that allows the patient to become comfortable with the tinnitus and defocuses their attention by utilizing sound generators such as hearing aids or even desktop devices such as fans to emit sounds at a lower level which still allow the user to hear the tinnitus with the intent of retraining the user's brain to eventually disregard the tinnitus. With habituation, a much lower level of sound therapy which does not mask the sound is delivered to the patient. In combination with therapy, habituation calms the patient and reinforces to them that their tinnitus is not life threatening or dangerous. Moreover, this therapy is meant to prevent the limbic system from increasing their awareness of and focus on Tinnitus. However, masking and TRT therapies may utilize conventional hearing aid devices which may be uncomfortable to the user and/or may carry other psychological stigmas. Additionally, in the case of TRT, such a therapy may take several years to accomplish.

Other devices such as cochlear implants and electrical stimulation, where an electrode array is inserted into the cochlea and a receiver is implanted subcutaneously behind the ear, may also be utilized to mask the tinnitus by ambient sounds and/or electrical stimulation. However, such procedures involve surgery and the complications typically associated therewith. Furthermore, drug therapy such as the use of antidepressants, may be effective in treating tinnitus. However, the typical side effects of ingesting such drugs may be highly undesirable to the tinnitus patient.

Accordingly, there exists a need for methods and devices for non-invasively and efficiently treating tinnitus patients.

### SUMMARY OF THE INVENTION

Tinnitus is a condition in which sound is perceived in one or both ears or in the head when no external sound is present. Such a condition may typically be treated by masking the tinnitus via a generated noise or sound. In one variation, the frequency or frequencies of the tinnitus may be determined through an audiology examination to pinpoint the range(s) in which the tinnitus occurs in the patient. This frequency or frequencies may then be programmed into a removable oral device which is configured to generate sounds which are conducted via the user's tooth or bones to mask the tinnitus.

An electronic and transducer device may be attached, adhered, or otherwise embedded into or upon the removable oral appliance or other oral device to form a hearing aid and/or sound generating assembly. Such an oral appliance may be a custom-made device fabricated through a variety of different process utilizing, e.g., a replicate model of a dental structure obtained by any number of methods. The oral appliance may accordingly be created to fit, adhere, or be otherwise disposed upon a portion of the patient's dentition to maintain the electronics and transducer device against the patient's dentition securely and comfortably.

The electronic and transducer assembly may be programmed to generate sounds at one or more frequencies depending upon the condition of the user's tinnitus via a vibrating transducer element coupled to a tooth or other bone structure, such as the maxillary, mandibular, or palatine bone structure. Moreover, the assembly may also be optionally configured to receive incoming sounds either directly or through a receiver to process and amplify the signals and transmit the processed sounds. Sound (e.g. Any tone, music, or treatment using a wide-band or narrow-band noise) generated via an actuatable transducer is calibrated and equalized to compensate for impedances of the teeth and bone.

One method for treating tinnitus may generally comprise masking the tinnitus where at least one frequency of sound (e.g., any tone, music, or treatment using a wide-band or narrow-band noise) is generated via an actuatable transducer positioned against at least one tooth such that the sound is transmitted via vibratory conductance to an inner ear of the patient, whereby the sound completely or at least partially masks the tinnitus perceived by the patient. In generating a wide-band noise, the sound level may be raised to be at or above the tinnitus level to mask not only the perceived tinnitus but also other sounds. Alternatively, in generating a narrow-band noise, the sound level may be narrowed to the specific frequency of the tinnitus such that only the perceived tinnitus is masked and other frequencies of sound may still be perceived by the user.

Another method may treat the patient by habituating the patient to their tinnitus where the actuatable transducer may be vibrated within a wide-band or narrow-band noise targeted to the tinnitus frequency perceived by the patient overlayed upon a wide-frequency spectrum sound. This wide-frequency spectrum sound, e.g., music, may extend over a range which allows the patient to periodically hear their tinnitus through the sound and thus defocus their attention to the tinnitus.

In enhancing the treatment for tinnitus, a technician, audiologist, physician, etc., may first determine the one or more frequencies of tinnitus perceived by the patient. Once the one or more frequencies have been determined, the audiologist or physician may determine the type of treatment to be implemented, e.g., masking or habituation. Then this information may be utilized to develop the appropriate treatment and to compile the electronic treatment program file which may be transmitted, e.g., wirelessly, to a processor coupled to the actuatable transducer such that the transducer is programmed to vibrate in accordance with the treatment program.

In use, an oral appliance containing the transducer may be placed against one or more teeth of the patient and the transducer may be actuated by the user when tinnitus is perceived to generate the one or more frequencies against the tooth or teeth. The generated vibration may be transmitted via vibratory conductance through the tooth or teeth and to the inner ear of the patient such that each of the frequencies of the perceived tinnitus is masked completely or at least partially.

The oral appliance may be programmed with a tinnitus treatment algorithm which utilizes the one or more frequencies for treatment. This tinnitus treatment algorithm may be uploaded to the oral appliance wirelessly by an external programming device to enable the actuator to vibrate according to the algorithm for treating the tinnitus. Moreover, the oral appliance may be used alone for treating tinnitus or in combination with one or more hearing aid devices for treating patients who suffer not only from tinnitus but also from hearing loss.

### BRIEF DESCRIPTION OF THE DRAWINGS

FIG. 1 illustrates the dentition of a patient's teeth and one variation of a hearing aid and/or sound generating assembly which is removably placed upon or against the patient's tooth or teeth as a removable oral appliance.

FIG. 2A illustrates a perspective view of the lower teeth showing one exemplary location for placement of the removable oral appliance hearing aid and/or sound generating assembly.

FIG. 2B illustrates another variation of the removable oral appliance in the form of an appliance which is placed over an entire row of teeth in the manner of a mouthguard.

FIG. 2C illustrates another variation of the removable oral appliance which is supported by an arch.

FIG. 2D illustrates another variation of an oral appliance configured as a mouthguard.

FIG. 3 illustrates a detail perspective view of the oral appliance positioned upon the patient's teeth utilizable in combination with a transmitting assembly external to the mouth and wearable by the patient in another variation of the device.

FIG. 4 shows an illustrative configuration of the individual components in a variation of the oral appliance device having an external transmitting assembly with a receiving and transducer assembly within the mouth.

FIG. 5 shows an illustrative configuration of another variation of the device in which the entire assembly is contained by the-oral appliance within the user's mouth.

FIG. 6A shows yet another illustrative variation of the device in which the sound generating device may be connected to a receiver for receiving programming signals to treat patient-specific tinnitus conditions.

FIG. 6B shows an example where the device assembly may be actuated via a separate transmitter assembly to control the operation of the device.

FIG. 7 illustrates a variation of one method for obtaining frequencies associated with tinnitus and which are patient-specific for programming an oral appliance.

FIG. 8A illustrates several variations for programming the electronics and/or transducer assembly with patient-specific tinnitus frequency or frequencies.

FIG. 8B schematically illustrates a variation where the electronics are separated from the transducer assembly.

FIGS. 9A and 9B illustrate examples for automatically decreasing the amplification level of generated sounds for treating tinnitus.

FIGS. 10A and 10B illustrate additional examples for automatically decreasing or terminating generated sounds for treating tinnitus.

FIG. 11 illustrates another example for decreasing generated sounds over a period of days, weeks, months, etc. to facilitate the withdrawal of a user's dependence on the masking noise.

FIGS. 12A and 12B illustrate an example for transmitting and correlating a patient's heart rate to masking sounds generated by an oral appliance.

FIGS. 13A and 13B illustrate top views of variations of a retainer integrated with one or more transducer assemblies.

FIG. 14A shows an example of a tinnitus treatment system using an external device for wirelessly transmitting a tinnitus treatment algorithm to a retainer assembly.

FIG. 14B shows another example of a tinnitus treatment system using the retainer assembly in conjunction with a hearing aid device.

FIG. 15 shows another example of a combination therapy system utilizing a microphone and a tinnitus treatment algorithm for transmission of auditory signals and treatment algorithm to the retainer assembly.

### DETAILED DESCRIPTION OF THE INVENTION

Because tinnitus is a condition in which sound is perceived in one or both ears or in the head when no external sound is present, such a condition may typically be treated by masking the tinnitus via a generated noise or sound. In one variation, the frequency or frequencies of the tinnitus may be determined through an audiology examination to pinpoint the range(s) in which the tinnitus occurs in the patient. This frequency or frequencies may then be programmed into a removable oral device which is configured to generate sounds which are conducted via the user's tooth or bones to mask the tinnitus, as described in further detail below.

An electronic and transducer device may be attached, adhered, or otherwise embedded into or upon the removable oral appliance or other oral device to form a hearing aid and/or sound generating assembly. Such an oral appliance may be a custom-made device fabricated through a variety of different process utilizing, e.g., a replicate model of a dental structure obtained by any number of methods. The oral appliance may accordingly be created to fit, adhere, or be otherwise disposed upon a portion of the patient's dentition to maintain the electronics and transducer device against the patient's dentition securely and comfortably.

The electronic and transducer assembly may be programmed to generate sounds at one or more frequencies depending upon the condition of the user's tinnitus via a vibrating transducer element coupled to a tooth or other bone structure, such as the maxillary, mandibular, or palatine bone structure. Moreover, the assembly may also be optionally configured to receive incoming sounds either directly or through a receiver to process and amplify the signals and transmit the processed sounds. Any tone, music, or treatment using a wide-band and or narrow band noise is calibrated and equalized to compensate for impedances of the tooth and bone and then that sound is generated via the actuatable transducer. Calibration and equalization can be done using several approaches. One approach is to use average impedance among a group of subjects representative of the targeted population. Another approach is to customize the calibration and equalization by obtaining the teeth and bone impedances for each patient.

Moreover, the electronic and transducer assembly may be configured to provide several different tinnitus treatments. For instance, the assembly may be configured to provide tinnitus masking therapy by providing sounds through bone conduction at a level and frequency that completely or partially cover the sounds of tinnitus to provide immediate short-term relief. Any tone, music, or treatment using a wide-band or narrow-band noise may be generated via the actuatable transducer positioned against at least one tooth such that the sound is transmitted via vibratory conductance to an inner ear of the patient, whereby the sound completely or at least partially masks the tinnitus perceived by the patient.

Alternatively, the assembly may be configured to provide habituation treatment, where the assembly provides sounds which may not mask the tinnitus but allows the patient to defocus their attention. The actuatable transducer may be vibrated within a wide-band or narrow-band noise targeted to the tinnitus frequency perceived by the patient overlayed upon a wide-frequency spectrum sound. This wide-frequency spectrum sound, e.g., music, may extend over a range which allows the patient to periodically hear their tinnitus through the sound and thus defocus their attention to the tinnitus.

Typically, this involves having a patient or treatment provider select a pleasant monaural piece of music having large fluctuations. The level fluctuations are preferably chosen to allow for the intermittent perception of the tinnitus by the patient, i.e., the tinnitus may be perceived by the patient during quiet passages in the music. A broadband, e.g., 14 kHz, white noise may be added or overlayed upon the music at a level that just masks the tinnitus yet still allows the music to be heard. The treatment provider may add amplification to the music and/or broadband white noise, e.g., via a graphic equalizer, to compensate for any hearing loss by the patient.

Taking this music and overlayed broadband white noise, an electronic stereo file may be produced from the monaural file where the same monaural file is used in each channel to equalize the phase. This treatment file may then be played by the patient, e.g., through an electronic music player and/or transmitted through the transducer.

In any of the treatment mechanisms or devices, either masking or habituation treatment may be effected by the assemblies described herein.

In yet another tinnitus treatment method similar to acoustic echo cancellation, an audiologist or physician may determine the tinnitus frequency perceived by a patient. With the frequency or frequencies known, a treatment signal may be generated, e.g., 5 kHz at 6 dB, which is shifted out-of-phase from the tinnitus frequencies, e.g., ideally 180° out-of-phase. This shifted treatment signal may be transmitted to a processor which actuates the transducer to vibrate the out-of-phase treatment signal through the patient's tooth, teeth, or bone structures such that the summation of the treatment signal with the tinnitus results in a cancellation of the tinnitus noise as perceived by the patient. Examples and further details of signal cancellation methods are described in U.S. Pat. app. Ser. No. 11/672,239 filed Feb. 7, 2007, which is incorporated herein by reference in its entirety.

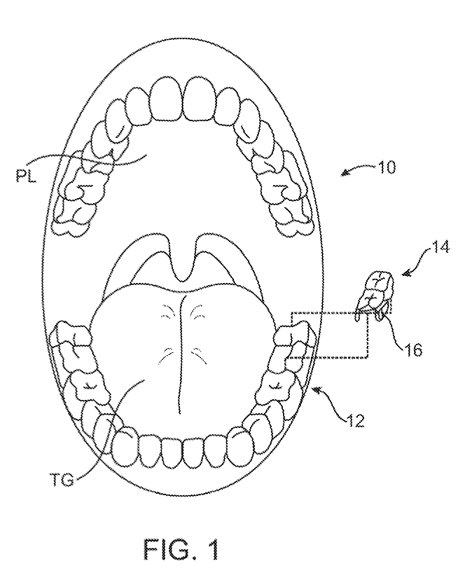

As shown in FIG. 1, a patient's mouth and dentition 10 is illustrated showing one possible location for removably attaching hearing aid and/or sound generating assembly 14 upon or against at least one tooth, such as a molar 12. The patient's tongue TG and palate PL are also illustrated for reference. An electronics and/or transducer assembly 16 may be attached, adhered, or otherwise embedded into or upon the assembly 14, as described below in further detail.

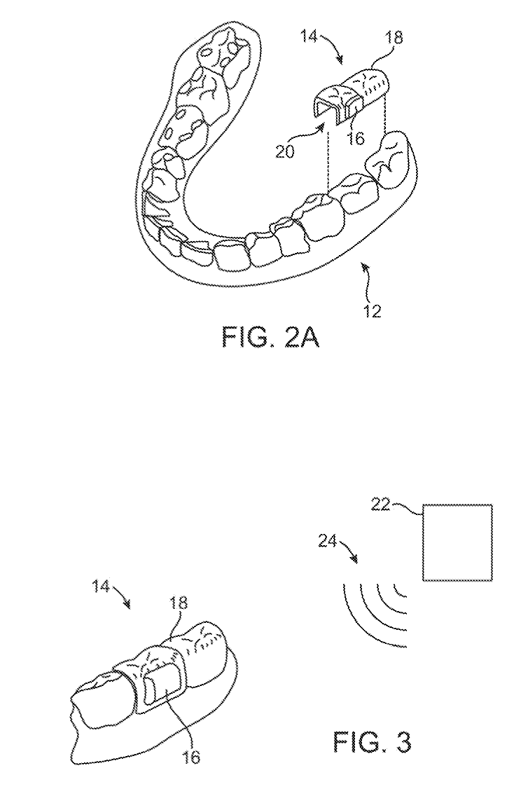

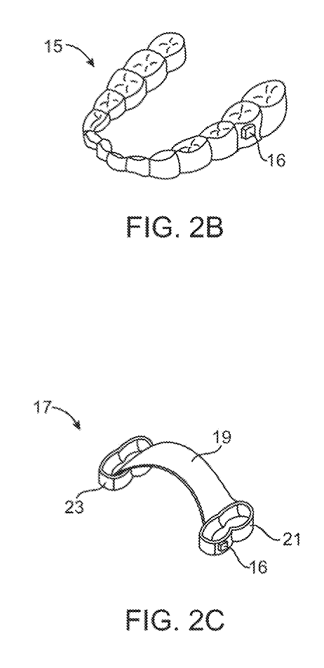

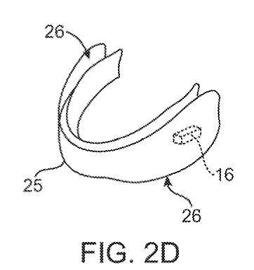

FIG. 2A shows a perspective view of the patient's lower dentition illustrating the hearing aid and/or sound generating assembly 14 comprising a removable oral appliance 18 and the electronics and/or transducer assembly 16 positioned along a side surface of the assembly 14. In this variation, oral appliance 18 may be fitted upon two molars 12 within tooth engaging channel 20 defined by oral appliance 18 for stability upon the patient's teeth, although in other variations, a single molar or tooth may be utilized. Alternatively, more than two molars may be utilized for the oral appliance 18 to be attached upon or over. Moreover, electronics and/or transducer assembly 16 is shown positioned upon a side surface of oral appliance 18 such that the assembly 16 is aligned along a buccal surface of the tooth 12; however, other surfaces such as the lingual surface of the tooth 12 and other positions may also be utilized. The figures are illustrative of variations and are not intended to be limiting; accordingly, other configurations and shapes for oral appliance 18 are intended to be included herein.

FIG. 2B shows another variation of a removable oral appliance in the form of an appliance 15 which is placed over an entire row of teeth in the manner of a mouthguard. In this variation, appliance 15 may be configured to cover an entire bottom row of teeth or alternatively an entire upper row of teeth. In additional variations, rather than covering the entire rows of teeth, a majority of the row of teeth may be instead be covered by appliance 15. Assembly 16 may be positioned along one or more portions of the oral appliance 15.

FIG. 2C shows yet another variation of an oral appliance 17 having an arched configuration. In this appliance, one or more tooth retaining portions 21, 23, which in this variation may be placed along the upper row of teeth, may be supported by an arch 19 which may lie adjacent or along the palate of the user. As shown, electronics and/or transducer assembly 16 may be positioned along one or more portions of the tooth retaining portions 21, 23. Moreover, although the variation shown illustrates an arch 19 which may cover only a portion of the palate of the user, other variations may be configured to have an arch which covers the entire palate of the user.

FIG. 2D illustrates yet another variation of an oral appliance in the form of a mouthguard or retainer 25 which may be inserted and removed easily from the user's mouth. Such a mouthguard or retainer 25 may be used in sports where conventional mouthguards are worn; however, mouthguard or retainer 25 having assembly 16 integrated therein may be utilized by persons, hearing impaired or otherwise, who may simply hold the mouthguard or retainer 25 via grooves or channels 26 between their teeth for receiving instructions remotely and communicating over a distance.

Generally, the volume of electronics and/or transducer assembly 16 may be minimized so as to be unobtrusive and as comfortable to the user when placed in the mouth. Although the size may be varied, a volume of assembly 16 may be less than 800 cubic millimeters. This volume is, of course, illustrative and not limiting as size and volume of assembly 16 and may be varied accordingly between different users.

In one variation configured as a hearing aid device, with assembly 14 positioned upon the teeth, as shown in FIG. 3, an extra-buccal transmitter assembly 22 located outside the patient's mouth may be utilized to receive auditory signals for processing and transmission via a wireless signal 24 to the electronics and/or transducer assembly 16 positioned within the patient's mouth, which may then process and transmit the processed auditory signals via vibratory conductance to the underlying tooth and consequently to the patient's inner ear.

The transmitter assembly 22, as described in further detail below, may contain a microphone assembly as well as a transmitter assembly and may be configured in any number of shapes and forms worn by the user, such as a watch, necklace, lapel, phone, belt-mounted device, etc.

Alternatively in another variation, transmitter assembly 22 may be configured as a transmitter for sending programming signals to electronics and/or transducer assembly 16 for programming specified frequencies or duration times for the transducer to vibrate, as described in further detail below.

In either case, in this and other variations, the transducer assembly 16 may generally be configured to have a frequency response of, e.g., 125 Hz to 20 kHz at 100 dB sound pressure level (SPL) peak and a frequency response of, e.g., 125 Hz to 1000 Hz based on uncomfortable vibration (UCV).

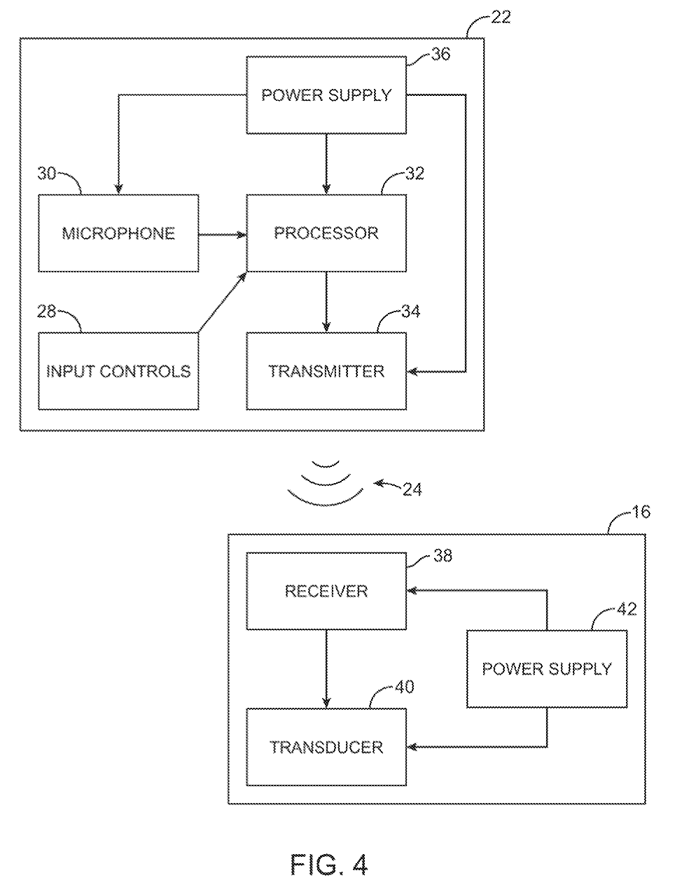

FIG. 4 illustrates a schematic representation of the variation where assembly 14 is configured as a hearing aid device utilizing an extra-buccal transmitter assembly 22, which may generally comprise microphone 30 for receiving sounds and which is electrically connected to processor 32 for processing the auditory signals. Processor 32 may be connected electrically to transmitter 34 for transmitting the processed signals to the electronics and/or transducer assembly 16 disposed upon or adjacent to the user's teeth. The microphone 30 and processor 32 may be configured to detect and process auditory signals in any practicable range, but may be configured in one variation to detect auditory signals ranging from, e.g., 125 Hertz to 20,000 Hertz.

With respect to microphone 30, a variety of various microphone systems may be utilized. For instance, microphone 30 may be a digital, analog, and/or directional type microphone. Such various types of microphones may be interchangeably configured to be utilized with the assembly, if so desired.

Power supply 36 may be connected to each of the components in transmitter assembly 22 to provide power thereto. The transmitter signals 24 may be in any wireless form utilizing, e.g., radio frequency, ultrasound, microwave, Blue Tooth® (BLUETOOTH SIG, INC., Bellevue, Wash.), etc. for transmission to assembly 16. Assembly 22 may also optionally include one or more input controls 28 that a user may manipulate to adjust various acoustic parameters of the electronics and/or transducer assembly 16, such as acoustic focusing, volume control, filtration, muting, frequency optimization, sound adjustments, and tone adjustments, etc.

The signals transmitted 24 by transmitter 34 may be received by electronics and/or transducer assembly 16 via receiver 38, which may be connected to an internal processor for additional processing of the received signals. The received signals may be communicated to transducer 40, which may vibrate correspondingly against a surface of the tooth to conduct the vibratory signals through the tooth and bone and subsequently to the middle ear to facilitate hearing of the user. Transducer 40 may be configured as any number of different vibratory mechanisms. For instance, in one variation, transducer 40 may be an electromagnetically actuated transducer. In other variations, transducer 40 may be in the form of a piezoelectric crystal having a range of vibratory frequencies, e.g., between 250 Hz to 14,000 Hz.

Power supply 42 may also be included with assembly 16 to provide power to the receiver, transducer, and/or processor, if also included. Although power supply 42 may be a simple battery, replaceable or permanent, other variations may include a power supply 42 which is charged by inductance via an external charger, e.g., every 24 hours. Additionally, power supply 42 may alternatively be charged via direct coupling to an alternating current (AC) or direct current (DC) source. Other variations may include a power supply 42 which is charged via a mechanical mechanism, such as an internal pendulum or slidable electrical inductance charger as known in the art, which is actuated via, e.g., motions of the jaw and/or movement for translating the mechanical motion into stored electrical energy for charging power supply 42. Moreover, the power supply 42 may be disposable where either the power supply 42 itself (if removable) or the entire assembly 16 may be disposed and replaced by a new assembly periodically, e.g., every 4 weeks.

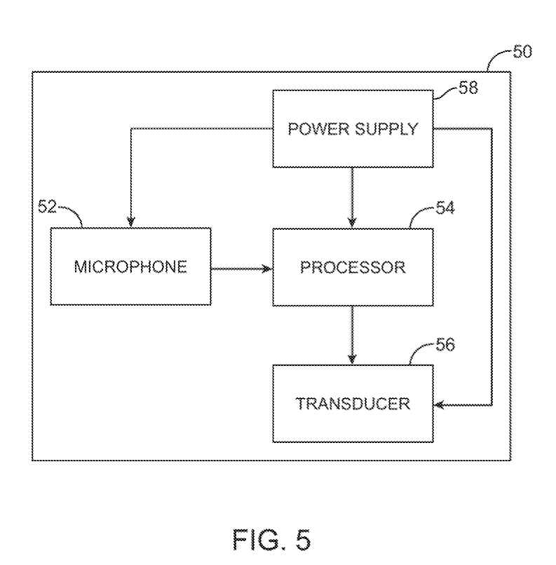

In another variation of assembly 16, rather than utilizing an extra-buccal transmitter, hearing aid assembly 50 may be configured as an independent assembly contained entirely within the user's mouth, as shown in FIG. 5. Accordingly, assembly 50 may include an internal microphone 52 in communication with an on-board processor 54. Internal microphone 52 may comprise any number of different types of microphones, as described above. Processor 54 may be used to process any received auditory signals for filtering and/or amplifying the signals and transmitting them to transducer 56, which is in vibratory contact against the tooth surface. Power supply 58, as described above, may also be included within assembly 50 for providing power to each of the components of assembly 50 as necessary.

The removable oral appliance 18 may be fabricated from various polymeric or a combination of polymeric and metallic materials using any variety of methods. For instance, in one variation of fabricating an oral appliance, a three-dimensional digital scanner may be used to image the dentition of the patient, particularly the tooth or teeth upon or about which the oral appliance is to be positioned. The scanned image may be processed via a computer to create a three-dimensional virtual or digital model of the tooth or teeth.

Various three-dimensional scanning modalities may be utilized to create the three-dimensional digital model. For instance, intra-oral cameras or scanners using, e.g., laser, white light, ultrasound, mechanical three-dimensional touch scanners, magnetic resonance imaging (MRI), computed tomography (CT), other optical methods, etc., may be utilized.

Once the three-dimensional image has been captured, the image may then be manipulated via conventional software to create a direct three-dimensional print of the model. Alternatively, the image may be used to directly machine the model. Systems such as computer numerical control (CNC) systems or three-dimensional printing processes, e.g., stereolithography apparatus (SLA), selective laser sintering (SLS), and/or other similar processes utilizing three-dimensional geometry of the patient's dentition may be utilized.

In another alternative, a mold may be generated from the print to then allow for thermal forming of the appliance directly upon the created mold. And yet in other variations, the three-dimensional image may be used to create an injection mold for creating the appliance.

In another variation of the device configured to additionally treat tinnitus instead of or in combination with treating hearing loss, sound generating assembly 60 may optionally contain a receiver 62 for receiving programming signals 24 from transmitter 34.

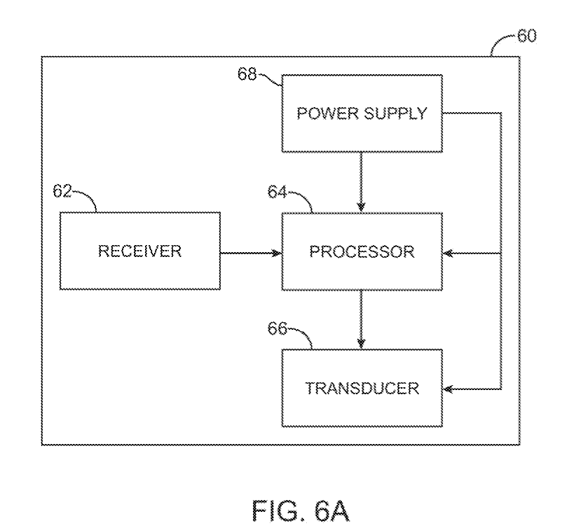

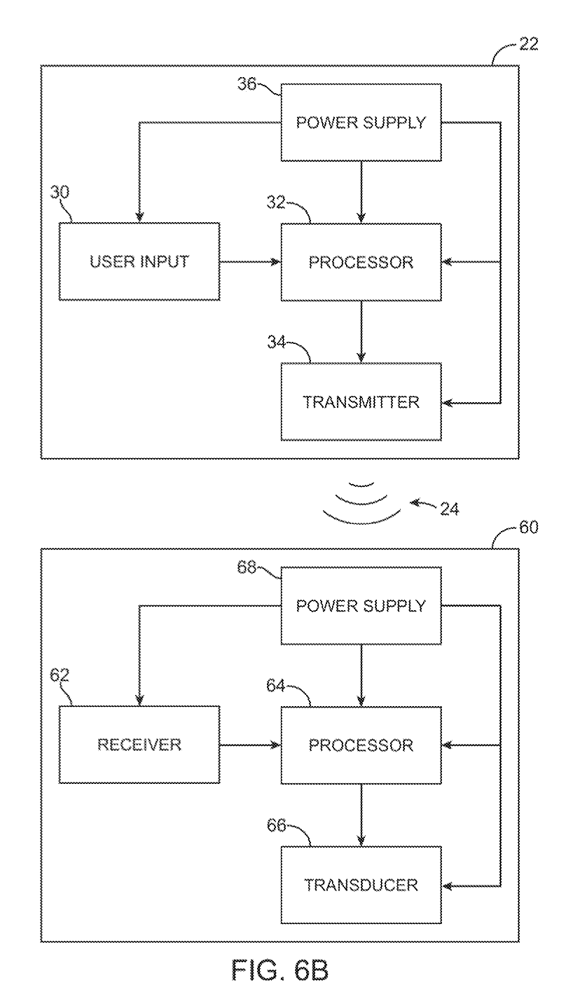

Receiver 62 may be in electrical communication with processor 64, powered by power supply 68, which in turn is electrically coupled to transducer 66, as shown in the schematic representation of FIG. 6A.

Power supply 68 may provide power to the receiver 62, transducer 66, and/or processor 64. Although power supply 68 may be a simple battery, replaceable or permanent, other variations may include a power supply 68 which is charged by inductance via an external charger. Additionally, power supply 68 may alternatively be charged via direct coupling to an alternating current (AC) or direct current (DC) source. Other variations may include a power supply 68 which is charged via a mechanical mechanism, such as an internal pendulum or slidable electrical inductance charger as known in the art, which is actuated via, e.g., motions of the jaw and/or movement for translating the mechanical motion into stored electrical energy for charging power supply 68.

In the variation where the sound generating assembly 60 is configured to function solely as a sound generating device to mask tinnitus, receiver 62 may be omitted from assembly 60 and transducer 66 may be configured to vibrate at a predetermined frequency or over a range of predetermined frequencies, e.g., anywhere from 250 Hz to 14,000 Hz, for a predetermined period of time, e.g., on the order of a few minutes up to several hours, as desired. The assembly may be accordingly actuated by the user on demand when desired to mask the tinnitus such that the transducer 66 vibrates, e.g., anywhere from 250 Hz to 14,000 Hz, for a specified preset time period or until deactivated by the user.

In the variation illustrated in FIG. 6B, assembly 60 may be actuated via transmitter assembly 22, as described above, to control the operation of the assembly 60. The transmitter signals 24 may be in any wireless form utilizing, e.g., radio frequency, ultrasound, microwave, Blue Tooth® (BLUETOOTH SIG, INC., Bellevue, Wash.), etc. for transmission to assembly 60. Assembly 22 may also optionally include one or more input controls 30 that a user may manipulate to turn the assembly 60 on or off as well as to optionally adjust various acoustic parameters of the electronics and/or transducer assembly 16, such as acoustic focusing, volume control, filtration, muting, frequency optimization and/or selection, sound adjustments, tone adjustments, time of operation or time delay of the transducer, etc.

Additionally, user input controls 30 may also include a feature to program and control the automatic activation or de-activation of the transducer 66 at preset times throughout the day, e.g., such as an alarm feature to automatically awake the user at a selected time or to automatically activate the transducer 66 at a selected time prior to or during the user's bedtime to automatically mask completely or partially the tinnitus.

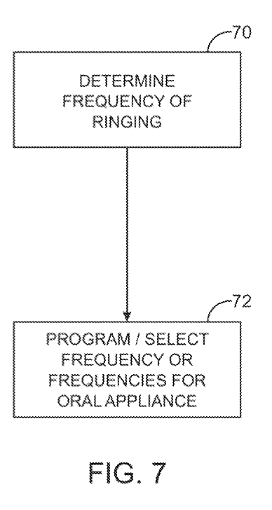

In an alternative variation, the assembly 60 may be configured to receive programming signals received by receiver 62. In such a variation, the device may be specifically programmed to vibrate the transducer 66 at specified frequencies and/or for specified periods of time which may be customized to patient-specific tinnitus conditions. Accordingly, the patient may be examined, e.g., by a technician, audiologist, physician, etc., to initially determine the frequency or frequencies of the tinnitus perceived by the patient 70, as indicated in FIG. 7, utilizing any audiology instruments or procedures such as tuning forks, audiometry, etc.

Once the patient-specific tinnitus frequency or frequencies have been determined, these frequency values may be programmed for an oral appliance 72 such that the transducer 66 may vibrate at the specified frequency or frequencies to optimally mask, or at least partially mask, the tinnitus. Alternatively, if the detected frequency or frequencies of tinnitus fall within certain frequency ranges, the oral appliance assembly 60 may be configured simply to vibrate the transducer 66 within preset frequency ranges rather than specific targeted frequency values.

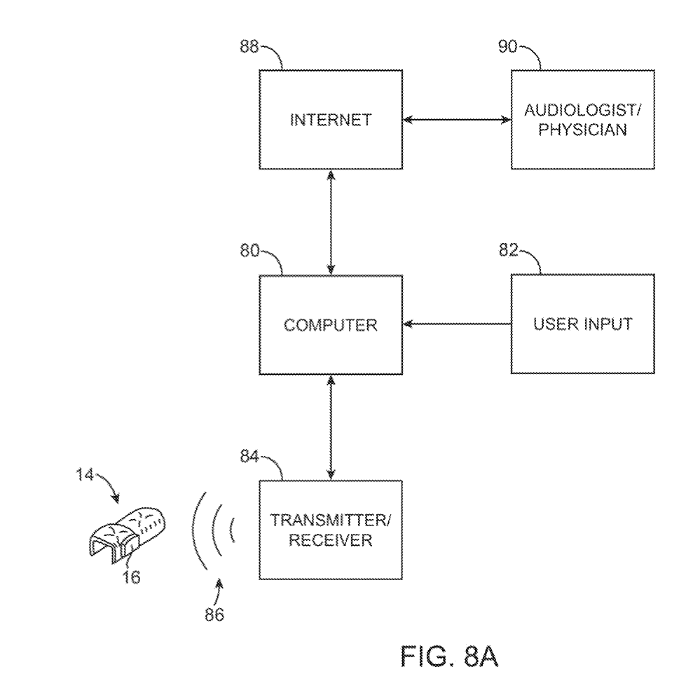

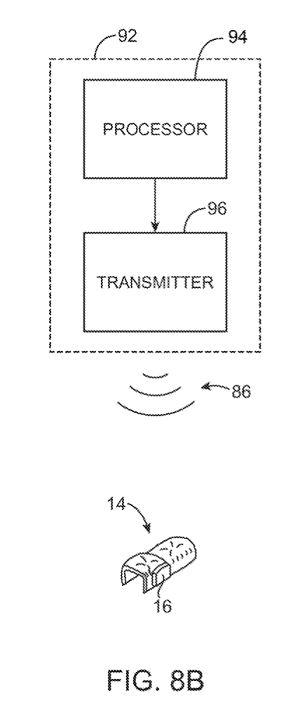

In order to program the electronics and/or transducer assembly 16 with patient-specific tinnitus frequency or frequencies, several alternative methods may be utilized to appropriately program the assembly 16, as illustrated in FIG. 8A. For instance, a technician, audiologist, physician, etc. may directly program the assembly 16 with a computer 80 in communication with a transmitter 84 to wirelessly transmit programming information 86 to receiver 62 contained within assembly 16.

Alternatively, a user may directly input 82 patient-related frequency information via a computer 80 to transmit the programming information 86 to assembly 16 via transmitter 84. In yet another variation, computer 80 may be connected to the internet 88 through which a technician, audiologist, physician, etc. 90 may input and/or access patient-specific frequency information for transmission to computer 80, which may then be used to transmit the information via transmitter 84 to assembly 16. Transmitter 84 may also be utilized as a receiver to optionally receive patient-specific information from assembly 16, in which case a transmitter may be incorporated into assembly 16.

In another variation for treating tinnitus, the electronics may be separated from the transducer assembly 16 to provide for a potentially smaller and less intrusive device 14 for delivering a masking treatment to the patient. As schematically illustrated in FIG. 8B, a base unit 92 may incorporate the electronics, including at least processor 94 and transmitter 96, to wirelessly transmit programming information 86 to the transducer assembly 16 for conductance to the patient. Base unit 92 may be configured into any number of different form factors, such as a base unit for placement on a nightstand or tabletop. Alternatively, base unit 92 may be configured for attachment onto a patient's belt much like a music player or IPOD device (Apple, Inc., Cupertino, Calif.). The transducer assembly 16 may contain a receiver for receiving the tinnitus masking or therapy programming information 86, a transducer for conducting the signals to the patient, and a power supply, as described above. In this and other variations where the transducer assembly 16 is configured to provide tinnitus habituation treatment, the programming information 86 may be combined or overlayed with music as selected by the user. Because other electronic components may be contained within base unit 92 rather than assembly 16, the device 14 may be configured into a relatively smaller configuration.

In other variations, rather than utilizing a device 14 which is placed within the mouth of a patient, assembly 16 may comprise an adhesive-backed assembly which may be temporarily attached at the entrance to the patient's ear canal and removed after use and disposed. In either case, the assembly 16 may be used by the patient at night prior to sleeping where base unit 92 may generate and wirelessly transmit the programming to the patient via device 14.

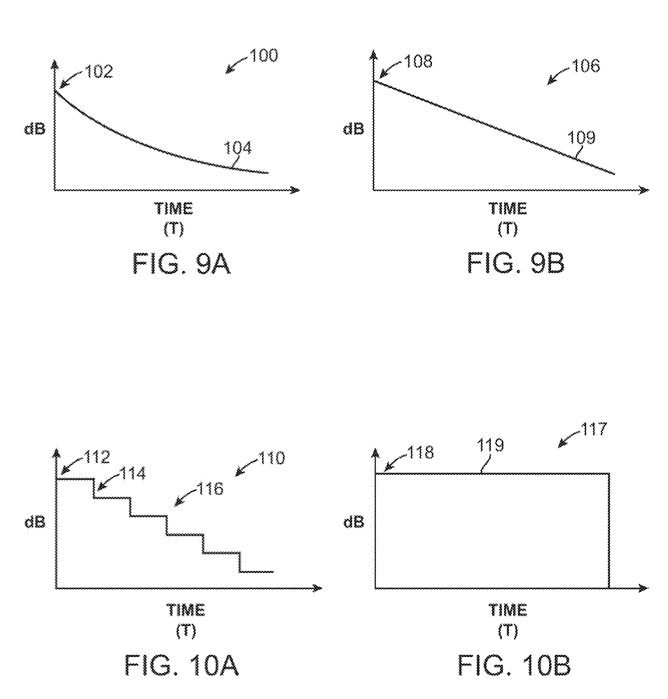

Aside from the ability to program specific frequencies into assembly 16 for which to vibrate the transducer 66, other patient-specific information such as periods of time for vibrating may also be programmed. Moreover, the amplification of the generated sound may also be eventually decreased automatically over this period of time in order to gradually decrease the user's dependence on the device, e.g., prior to and during the initial phases of sleep. In one variation, as shown in FIG. 9A, the transducer 66 may be programmed to vibrate at one or more specified frequencies upon actuation at a first starting decibel (dB) level 102, e.g., 30 dB to 40 dB. The first starting level 102 may be varied depending upon the user's tinnitus condition. Over a specified time period, T, e.g., anywhere from several minutes to several hours, the amplification level of the vibrating transducer 66 may be reduced exponentially 104, as shown in the plot 100 to gradually reduce the masking noise until the transducer 66 is stopped altogether. Alternatively, the amplification level may be reduced linearly, as shown in the plot 106 in FIG. 9B, such that the transducer 66 may begin at a first starting decibel level 108 upon actuation and gradually reduces linearly 109 until the transducer 66 is stopped altogether.

In these and other examples, although the levels are illustrated as decreasing over time, they may alternatively be increased for set periods of time intermittently or gradually over time, depending upon the desired treatment.

In another alternative, FIG. 10A illustrates a plot 110 where the amplification level may be stepped 116 such that the level begins at a first constant decibel level 112 and then after a period of time, T, steps down 114 to a second level. The amplification may be eventually stepped down in uniform (or non-uniform) increments until the transducer 66 is stopped altogether. In another variation, the amplification may begin at a first level 118 and maintain a constant level 119 for a period of time, T, until the device is stopped altogether, as shown in the plot 117 in FIG. 10B.

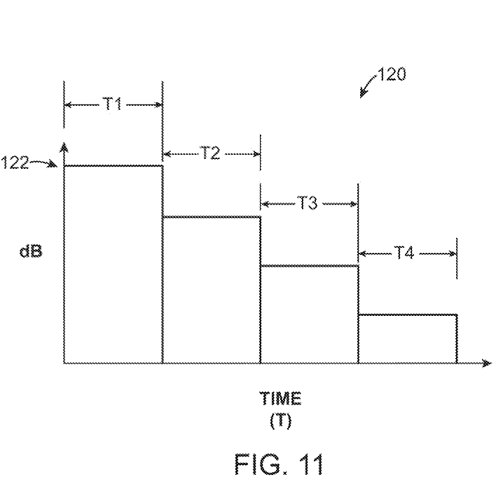

In yet another variation shown in the plot 120 of FIG. 11, the transducer 66 may be programmed to gradually decrease its amplification over a period of days, weeks, months, or even longer to facilitate the withdrawal of a user's dependence on the masking noise. For instance, the transducer 66 may initially provide a first dB level 122 for a first period of time T1. The assembly may then automatically reduce its amplification level over a second period of time T2, and eventually further reduce its amplification level over a third period of time T3, and another reduction over a fourth period of time T4, and so on, until the user no longer requires use of the device to tolerate his or her tinnitus condition. Each period of time may be programmed to range uniformly or otherwise anywhere from days to months or even longer depending upon the user's tinnitus condition.

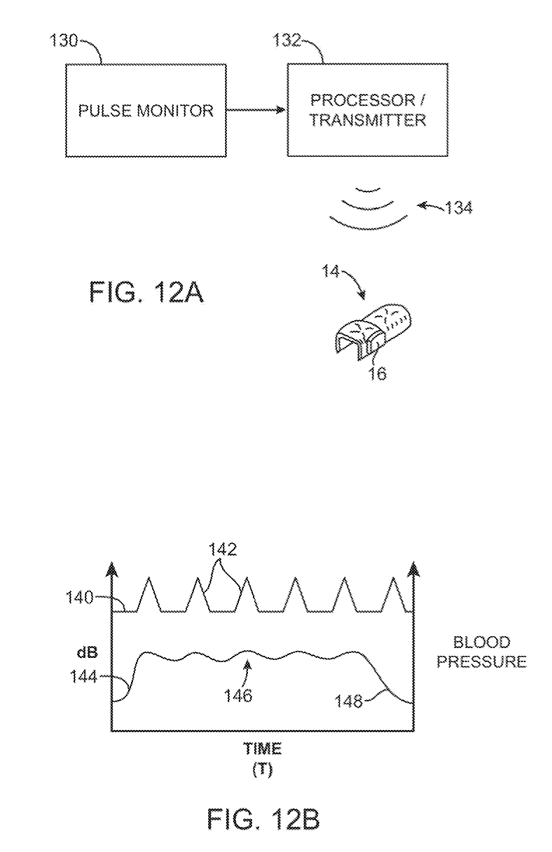

In yet another variation shown in FIGS. 12A and 12B, conditions such as pulsatile tinnitus, as mentioned above, may also be treated using the hearing aid and/or sound generating assembly 14. In patients suffering from pulsatile tinnitus, the sound associated with tinnitus typically occurs with a rhythmic pulsing sound attuned to the patient's heartbeat. Accordingly, the electronics and/or transducer assembly 16 within the assembly 14 may be programmed to produce a masking noise or to raise its amplification of ambient noise correspondingly with the patient's detected pulse. A pulse monitor 130 may be worn by the patient, e.g., around the wrist, and may be connected wirelessly or otherwise to processor/transmitter 132. The detected pulse may be processed by processor 132 and transmitted wirelessly 134 to hearing aid and/or sound generating assembly 14, as shown in FIG. 12A.

The received signals may be utilized by assembly 14 to raise its amplification dB level 144 and to maintain an elevated level 146 corresponding to the detected heart beat 142, as seen along plot 140, which corresponds to the detected heart beat, blood pressure, electrical activity, etc. of the patient's heart, as shown in FIG. 12B. After a preset or programmed period of time, as described above, the amplification level may be automatically decreased 148.

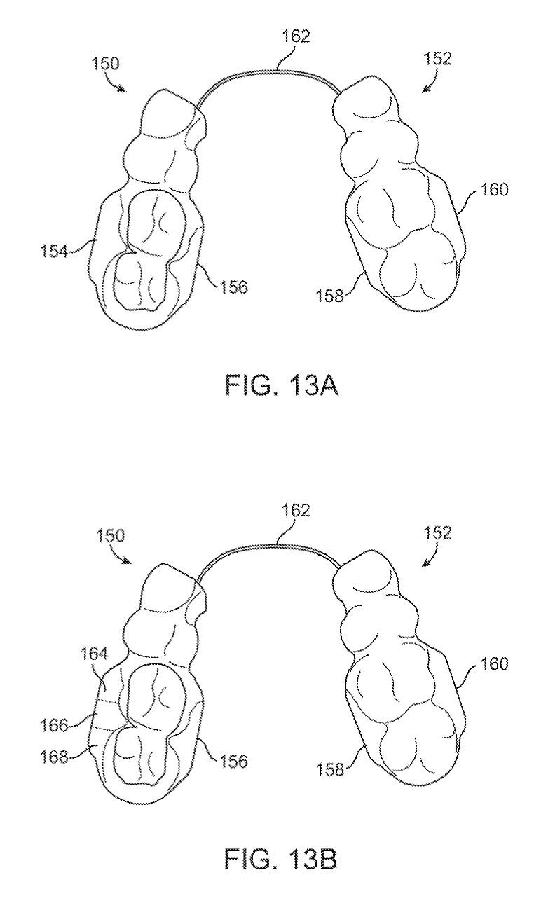

In yet another variation for delivering a tinnitus treatment to a patient, a configuration utilizing a connecting member 162 which may be positioned along the lingual or buccal surfaces of a patient's row of teeth to connect a first tooth retaining portion 150 and a second tooth retaining portion 152 is shown in FIG. 13A. One or more transducer assemblies 154, 156 may be integrated within the first retaining portion 150 to align along the buccal and lingual surfaces of one or more teeth. Similarly, one or more transducer assemblies 158, 160 may also be integrated within the second retaining portion 152 to align along the lingual and buccal surfaces of one or more teeth.

FIG. 13B illustrates a variation where at least one of the transducer assemblies integrated with a retaining portion 150 may be configured to include a battery 164, transducer 166, and associated electronics 168 such as a receiver, processor, and memory. The battery 164 may be a rechargeable or disposable type battery and the transducer 166 may be an electromagnetic transducer or a piezoelectric transducer. Piezoelectric transducers in particular may be used in various configurations due in part to the various vibrational modes which may be utilized to transmit the acoustic signals as vibrations through a tooth or teeth. Any number of transducers may be utilized for particular applications. For instance, low voltage multi-layer piezoelectric transducers manufactured by Morgan Electro Ceramics Ltd. (Wrexham, England) may be utilized for the applications described herein. Further examples of transducers and housing assemblies which may be utilized for the tinnitus treatments herein are shown and described in further detail in U.S. Pat. apps. 11/741,648 filed Apr. 27, 2007 and Ser. No. 11/754,823 filed May 29, 2007, each of which is incorporated herein by reference in its entirety.

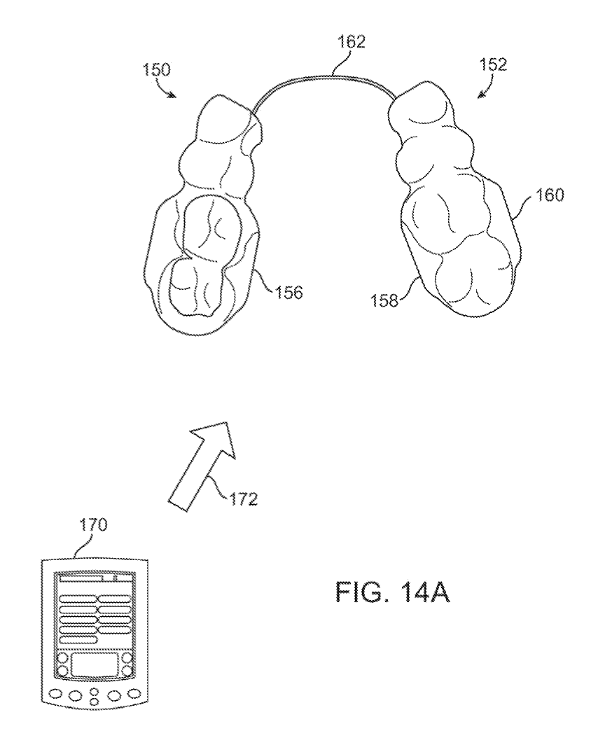

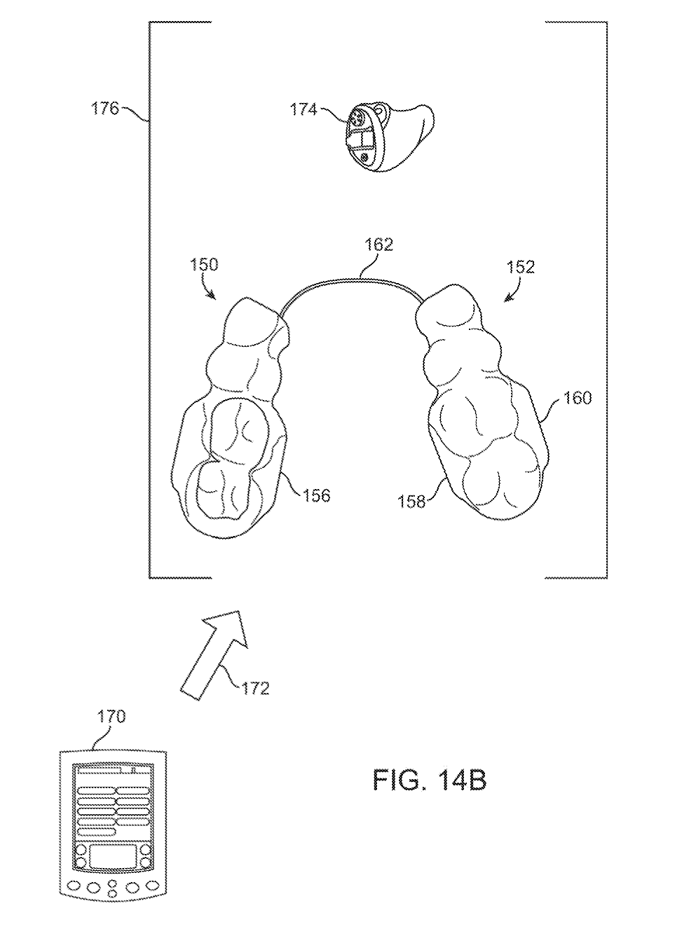

The system for tinnitus treatment can take a number of different forms. In one example, an external programming device 170 such as a PDA, cell phone, music player, etc. can be programmed to transmit 172, e.g., via RF or Blue Tooth® (BLUETOOTH SIG, INC., Bellevue, Wash.), etc. the sound therapy programming treatment algorithm to the retainer 150, as shown in FIG. 14A. For transmitting signals 172 from external device 170 to retainer 150, particularly when utilizing RF, the signals 172 may be transmitted in the range of 1 MHz to 6 GHz. As described above for FIG. 8B, external device 170 may be programmed by the clinician for specific algorithms of treatment with one or more programs (for sleep, work, during exercise, etc.) that the patient or physician can select via the device 170.

Additionally, the patient may control certain features of the external device 170 for enhanced comfort or additional programming features. For instance, the patient may control an ON/OFF selection, a volume of the treatment signal, program an alarm feature such that the treatment begins and/or ends at preselected times, program a sleep feature where the patient can program the retainer 150 to activate for a predetermined length of time before automatically shutting off, select desired sound files, etc. The external device 170 may also be programmed to upload selected files, retain a compliance indicator or data log of the times and duration which the patient used the retainer 150, and it may also utilize a power indicator to notify the user that either the external device 170 and/or retainer 150 is powered.

The physician can also lock the patient from making any adjustments in program choice or volume of the tinnitus treatment. In either case, the external device 170 may upload the selected treatment to the retainer 150 and download compliance information for storage for the physician to review for further treatment enhancement, if necessary. Moreover, external device 170 may carry its own battery power supply which may be recharged periodically, as described above, or simply re-supplied with a new power supply.

To maintain consistency and uniformity with industry standards, the external device 170 may be programmed to conform with NOAH, which is an industry standard supported by a framework of companies within the audiology industry. Adherence to this industrial standard may allow for the programming information as well as any audiological measurements to interface with a common database.

Other tinnitus treatment algorithms which utilize software to spectrally modify audio signals in accordance with predetermined masking algorithms which modify the intensity of the audio signal at selected frequencies may also be used. For instance, a masking algorithm may provide for intermittent masking of the tinnitus where, at listening levels, during peaks of the audio signal, such as music, the tinnitus is completely obscured while during troughs in the audio signal, the perception of tinnitus occasionally emerges. Such algorithms may be programmed and transmitted to the retainer 150 and transmitted via vibrational conductance to the patient's tooth or bone. Details of such algorithms are described in further detail in U.S. Pat. Pub. 2004/0141624 A1 filed Dec. 4, 2003, which is incorporated herein by reference in its entirety.

A certain number of patients who suffer from tinnitus also suffer from hearing loss. Upwards of 80% of the patients with tinnitus also have some form of hearing loss which is a significant issue in treating the tinnitus with a sound therapy device that is meant to provide tinnitus therapy while also allowing the patient to continue with his/her normal daily activities. One approach to compensating for the hearing loss while also treating tinnitus includes a combination treatment 176 utilizing the oral appliance 150 for treating the tinnitus along with a hearing aid 174 for treating the hearing loss. Oral appliance 150 may also compensate for the sensorineural hearing loss by increasing the tinnitus treatment signal itself by up to 40 dB for treating the tinnitus without increasing for the input hearing. Any tone, music, or treatment using a wide-band and or narrow band noise may also be calibrated and equalized to compensate for impedances of the tooth and bone as well as for the sensorineural hearing loss and then that sound may be generated via the actuatable transducer. Calibration and equalization can be done using several approaches. One approach is to use average impedance among a group of subjects representative of the targeted population. Another approach is to customize the calibration and equalization by obtaining the teeth and bone impedances for each patient.

Most conventional hearing aid devices 174 are typically worn outside the ear or within the ear canal of the patient and does not allow for use of a tinnitus treatment device. However, use of the oral appliance 150 worn upon the user's tooth or teeth does not interfere with the wearing of a hearing aid within the ear but can instead complement the use of the hearing aid in conjunction with the appliance 150, as shown in FIG. 14B.

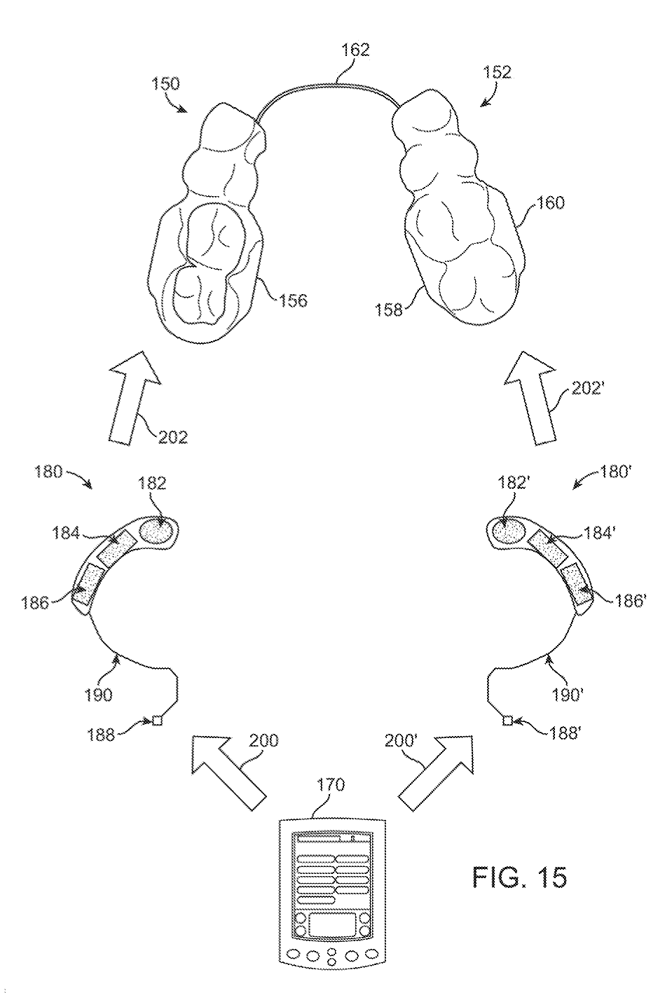

Another approach for treating patients having both tinnitus and hearing loss may include a combination therapy system 180 which may include a microphone 188 and receiver that would allow external sounds to be transmitted to the retainer device 150, amplified and then subsequently delivered to the cochlea via bone conduction, as described above. The combination system 180 may include a connecting wire 190 which electrically couples microphone 188 to system 180 which may also include a processor 184 and a wireless transmitter 186 which may both be powered by battery 182, as shown in FIG. 15. System 180 may amplify external sounds received by the user but it may also receive the tinnitus programming information 200 from an external device 170. Processor 184 may combine any perceived auditory signals detected by microphone 188 and combine the tinnitus programming information 200 received from external device 170 and transmit this combined information 202 to retainer 150 for vibratory conduction to the user.

Although a single therapy system 180 may be used, a second complementary system 180′ also containing a microphone 188′ coupled by wire 190′ to a processor 184′, a wireless transmitter 186, and battery 182′ may be utilized to also receive tinnitus programming information 200′ from external device 170 and to transmit 202′ any perceived auditory signals along with the tinnitus programming to the same or different retainer 160 for vibratory conduction to the user for treatment.

Additionally, in determining patient-specific frequency information, as described above, the assembly 16 can also be used to measure a patient's bone conductive hearing loss directly through the assembly 16 for determining appropriate gain levels for a patient's individual hearing loss profile. To measure the conductive hearing loss, the assembly 16 can be connected, wirelessly or wired, to a standard audiometer to measure the hearing threshold of the patient at the tooth or bone directly through assembly 16 by using standard audiometric protocols.

The patient may be asked to match their tinnitus sound level and frequency by listening to tones at different frequencies generated through the assembly 16 to first establish the tinnitus frequency. The frequency level may be gradually increased to match the tinnitus level perceived by the patient. With this correlated information, the assembly 16 and/or external device 170 may be programmed accordingly with the patient's hearing loss profile and adjusted for appropriate gain at each frequency during tinnitus treatment. Additionally, the programming software may configure a customized sound therapy treatment according to the physician's preference (e.g., wide or narrow band noise, recorded sounds, etc) based upon the patient's tinnitus frequency and level.

The applications of the devices and methods discussed above are not limited to the treatment of tinnitus and/or hearing loss but may include any number of further treatment applications. Moreover, such devices and methods may be applied to other treatment sites within the body. Modification of the above-described assemblies and methods for carrying out the invention, combinations between different variations as practicable, and variations of aspects of the invention that are obvious to those of skill in the art are intended to be within the scope of the claims.

What is claimed is:

1. An oral appliance system configured for bone conduction through a patient's dentition, comprising:
a first retaining portion adapted for placement against a first set of one or more teeth;
a second retaining portion adapted for placement against a second set of one or more teeth, wherein the first and second retaining portions are positioned at or along opposing regions of the patient's dentition in a bi-lateral configuration;
a coupling member connecting the first retaining portion and the second retaining portion such that the coupling member is positioned adjacent to a lingual or buccal surface of the patient's dentition; and
at least one transducer within or along one of the retaining portions such that the transducer is placed into vibratory contact against the one set of one or more teeth.

2. The system of claim 1 wherein the first and second retaining portions are conformable against respective first and second sets of one or more teeth.

3. The system of claim 1 wherein the coupling member is positioned adjacent to the lingual surfaces of an upper row of the patient's dentition.

4. The system of claim 1 wherein the at least one transducer is aligned along a buccal or lingual surface of one set of one or more teeth.

5. The system of claim 1 further comprising at least one additional transducer within or along the first retaining portion.

6. The system of claim 1 further comprising at least one additional transducer within or along the second retaining portion.

7. The system of claim 1 further comprising a power supply within the first retaining portion or second retaining portion.

8. The system of claim 1 further comprising a receiver within the first retaining portion or second retaining portion.

9. The system of claim 8 further comprising an external programming device in wireless communication with the appliance.

10. The system of claim 1 further comprising a microphone within the first retaining portion or second retaining portion and adapted to receive auditory signals for vibratory conductance via the transducer.

11. The system of claim 1 further comprising a hearing aid utilized in combination with the oral appliance.

12. An oral appliance system configured for bone conduction through a patient's dentition, comprising:
a first retaining portion which is conformable against a lingual and/or buccal surface of a first set of one or more teeth;
a second retaining portion which is conformable against a lingual and/or buccal surface of a second set of one or more teeth, wherein the first and second retaining portions are positioned at or along opposing regions of the patient's dentition in a bi-lateral configuration;
a coupling member connecting the first retaining portion and the second retaining portion such that the coupling member is positioned adjacent to a lingual and/or buccal surface of the patient's dentition; and
at least one transducer within or along one of the retaining portions such that the transducer is placed into vibratory contact against the lingual and/or buccal surface of one set of one or more teeth.

13. The system of claim 12 wherein the coupling member is positioned adjacent to the lingual surfaces of an upper row of the patient's dentition.

14. The system of claim 12 wherein the at least one transducer is aligned along a buccal surface of the first set of one or more teeth.

15. The system of claim 12 further comprising at least one additional transducer within or along the first retaining portion.

16. The system of claim 12 further comprising at least one additional transducer within or along the second retaining portion.

17. The system of claim 12 further comprising a power supply within the first retaining portion or second retaining portion.

18. The system of claim 12 further comprising a receiver within the first retaining portion or second retaining portion.

19. The system of claim 18 further comprising an external programming device in wireless communication with the appliance.

20. The system of claim 12 further comprising a microphone within the first retaining portion or second retaining portion and adapted to receive auditory signals for vibratory conductance via the transducer.
